(19) United States
(12) Patent Application Publication
(10) Pub. No.: US 2008/0039724 A1
(43) Pub. Date: Feb. 14, 2008

(54) ULTRASOUND TRANSDUCER WITH IMPROVED IMAGING

(76) Inventors: Ralf Seip, Indianapolis, IN (US); Narendra T. Sanghvi, Indianapolis, IN (US); Wo-Hsing Chen, Fishers, IN (US)

Correspondence Address: BAKER & DANIELS LLP, 300 NORTH MERIDIAN STREET, SUITE 2700, INDIANAPOLIS, IN, 46204, US

(21) Appl. No.: 11/463,692

(22) Filed: Aug. 10, 2006

(51) Int. Cl. A61B 8/00

(52) U.S. Cl. 600/439

(57) ABSTRACT

An acoustic transducer, and in particular to an ultrasound transducer, provides high intensity focused ultrasound (“HIFU”) therapy to tissue and images the tissue.

### BACKGROUND AND SUMMARY OF THE INVENTION

The present invention relates to an acoustic transducer, and in particular to an ultrasound transducer used to provide high intensity focused ultrasound (“HIFU”) therapy to tissue and to image tissue.

The treatment of tissue with HIFU energy is known in the art. Further, it is known to image the tissue being treated with an ultrasound transducer. In addition, it is known to use a single crystal, two-element transducer to both image the tissue and to provide the actual treatment of the tissue with HIFU.

An exemplary system for treating tissue with HIFU is the Sonablate®-500 system available from Focus Surgery located at 3940 Pendleton Way, Indianapolis, Ind. 46226. The Sonablate 500 system uses a dual-element, confocal ultrasound transducer which is moved by mechanical methods, such as motors, under the control of a controller. Typically one element of the transducer, the central element or electrode, is used for imaging and either the outer element only or both elements (the central and outer elements or electrodes) of the transducer are used for providing HIFU therapy to the tissue to be treated.

Ultrasound transducers typically include a transducer member, such as a piezo-electric crystal, which generates and/or detects acoustic energy. Both the transducer member and the surrounding environment have an associated acoustic impedance. Assuming that the acoustic impedance of transducer member is generally the same as the acoustic impedance of the surrounding environment, acoustic energy flows from the transducer member to the surrounding environment generally in its most efficient way. However, there is often a difference between the acoustic impedance of the transducer member and the acoustic impedance of the surrounding environment. This mismatch results in less acoustic energy being transferred from the transducer member to the surrounding environment. The reduction in transfer of acoustic energy results in the generation of heat associated with the transducer member which may lead to damage to transducer member or to the surrounding environment. Further, the reduction in transfer of acoustic energy results in a higher level of electric energy required to provide sufficient acoustic energy at a treatment site in the surrounding environment.

It is known to apply an acoustical matching layer to a front surface of the transducer member to reduce the acoustic impedance mismatch between transducer member and the surrounding environment. By reducing the acoustic impedance mismatch, less energy is required to provide therapy and less heat is generated at the transducer. Generally, the acoustical matching layer has an acoustic impedance value between the acoustic impedance of transducer member and the acoustic impedance of the surrounding environment.

The thickness of the matching layer is one factor in the performance of the transducer Two known methods are used in the manufacture of transducers to make sure an appropriate thickness matching layer is applied. These methods include the use of thickness gauges to measure the thickness of the matching layer at various positions of the transducer surface and the monitoring of the shape of an echo pulse received based on acoustic pulse emitted by the transducer.

Copending U.S. application Ser. No. 11/175,947, owned by the assignee of the present application and incorporated herein by reference, discloses a method for optimizing an ultrasound transducer for therapy applications. In one example, the ultrasound transducer is optimized to provide therapy with HIFU at a desired frequency by controlling characteristics of the matching layer applied to the front surface of a crystal of the transducer.

Conventional single crystal ultrasonic transducers use a single crystal for both imaging and HIFU treatment. This is accomplished by using a curved transducer element formed from a spherical shell of a fixed radius or focal length. Illustratively, a central circular portion (“center element”) of the transducer element having a predetermined diameter is used for imaging. Typical single crystal transducers have a single operating frequency, such as a frequency of about 4 MHz, for example, for both imaging and treatment modes of operation.

Transducers used in imaging applications typically operate at acoustic power levels of a few milliwatts. In contrast, transducers used for therapy applications are required to emit higher amounts of acoustic power than for traditional imaging applications, such as in the range of about 5 to more than 100 Watts.

The ultrasonic transducer of the present invention permits a higher frequency to be used for imaging than for therapy on a single crystal transducer. This higher operating frequency for an imaging mode of operation improves image quality for the transducer.

In prior art systems, in order to obtain a transducer assembly able to treat at one frequency and image at another frequency, a completely separate imaging transducer assembly with the desired imaging characteristics is mounted in a hole cut through the therapy crystal and matching layer. Having separate crystal thicknesses (and even materials), separate matching layers, and separate backing materials allows this optimization. This prior art system, however, is expensive, requires careful alignment between the focal zones of both imaging and therapy transducer assemblies (as they are no longer manufactured on the same crystal), requires careful waterproofing where both matching layers meet, and may not be cosmetically appealing and reliable as the single crystal transducer of the illustrated embodiments of the present invention.

In an illustrated embodiment, the matching layer applied to a front face of the crystal is optimized for the therapy mode of operation. The rear surface of the crystal opposite from the matching layer corresponding to the imaging portion of the transducer (center element) is formed to include a recessed portion which receives an imaging electrode therein. The front or outer surface of the transducer defined by the matching layer remains smooth. A therapy electrode (“outer element”) is located on the rear surface of the crystal surrounding the recessed portion. A controller is used to drive both the imaging and therapy electrodes. The ultrasonic transducer crystal forming the center imaging element now can oscillate at two different frequencies, one mainly defined by the imposed thickness of the matching layer and another one mainly defined by the reduced thickness of the crystal in the area of the imaging electrode. As long as both of these frequency modes are not significantly separated from each other, this provides a new overall frequency spectrum having a larger bandwidth and higher center frequency for the ultrasonic transducer of the present invention compared to conventional single crystal transducers.

An illustrated ultrasound transducer for providing HIFU therapy and imaging includes a crystal having a generally concave first surface and a generally convex second surface. The second surface of the crystal is formed to include a recessed portion. The transducer also includes a matching layer coupled to the first surface of the crystal. The matching layer has a smooth outer surface. The transducer further includes a therapy electrode coupled to the second surface of the crystal adjacent the recessed portion, and an imaging electrode located in the recessed portion formed in the second surface of the crystal.

An illustrated method of improving an image detected by an ultrasound transducer which provides HIFU therapy and imaging includes the steps of providing a crystal having a generally concave first surface and a generally convex second surface, and applying a matching layer to the first surface of the crystal to optimize a therapy function of the transducer. Illustratively, the matching layer has a smooth outer surface. the method also includes forming a recessed portion in the second surface of the crystal, positioning a therapy electrode on the second surface of the crystal adjacent the recessed portion, and positioning an imaging electrode within the recessed portion of the second surface of the crystal.

Another illustrated method of operating an ultrasound transducer to provide HIFU therapy and imaging includes the steps of providing a single crystal having a first surface and a second surface, oscillating the single crystal at a first frequency for a therapy function of the transducer, and oscillating the single crystal at a second frequency for an imaging function of the transducer, the second frequency being higher than the first frequency.

Additional features of the present invention will become apparent to those skilled in the art upon consideration of the following detailed description of illustrative embodiments exemplifying the best mode of carrying out the invention as presently perceived.

### BRIEF DESCRIPTION OF THE DRAWINGS

The detailed description of the drawings particularly refers to the accompanying figures in which:

FIG. 1 is an exploded perspective view of an ultrasound transducer having a single crystal transducer element, a matching layer, a therapy electrode, and an imaging electrode;

FIG. 2 is a sectional view taken through a prior art ultrasound transducer;

FIG. 3 is a diagrammatical sectional view taken through the transducer of FIG. 1 illustrating a recessed portion formed in a rear surface of the crystal for receiving the imaging electrode therein;

FIG. 4 is a diagrammatical view similar to FIG. 3 illustrating the imaging transducer located within the recessed portion of the crystal;

FIG. 5 a graph comparing a frequency spectrum of the prior art transducer of FIG. 2 with a frequency spectrum of one embodiment of the transducer of the present invention illustrated in FIGS. 1, 3 and 4;

FIGS. 6 and 7 are graphs illustrating frequency spectrums of various other configurations of transducers;

FIG. 8 is a graph illustrating an oscillation frequency of the crystal compared to a thickness of the crystal;

FIG. 9 is a tool used to form the recessed portion in the crystal; and

FIG. 10 is a diagrammatical view of another embodiment of the present invention.

### DETAILED DESCRIPTION OF THE DRAWINGS

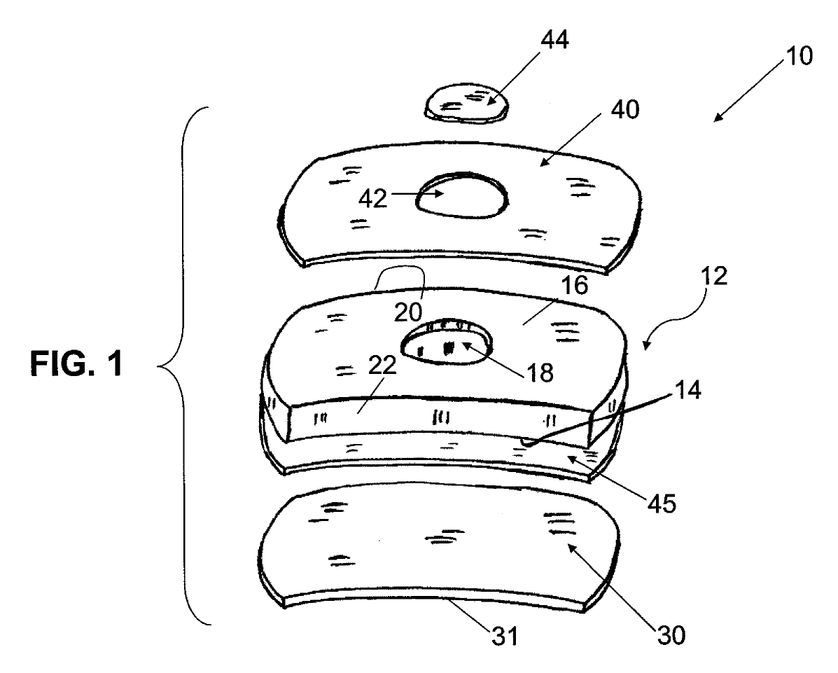

Referring to FIG. 1, a transducer 10 of an illustrated embodiment is shown. Transducer 10 includes a transducer member or crystal 12 capable of emitting an acoustic signal. Crystal 12 includes a first generally concave front surface 14 and a second generally convex rear surface 16. Illustratively, crystal 12 is a single crystal element made in a conventional manner. Crystal 12 may be an ultrasound crystal (i.e. piezoelectric crystal). It is understood that composite ultrasound transducers (i.e. piezocomposite structures) may also be used in accordance with certain aspects of the present invention. One illustrated transducer is shown in U.S. Pat. No. 5,117,832, which is incorporated herein by reference. Such a single crystal transducer is also used in the Sonablate-500® system available from Focus Surgery discussed above.

As discussed in more detail below, the second surface 16 of the crystal 12 is formed to include a recessed portion 18 therein. The recessed portion 18 is illustratively circular in shape and located in substantially a central portion of the crystal 12. It is understood that the recessed portion 18 may extend to the opposite side edges 20 and 22, if desired.

Ultrasound transducer 10 further includes a matching layer 30 which is applied to the first, generally concave front surface 14 of crystal 12. In one embodiment, matching layer 30 is an epoxy mixture applied to the surface 14. In another embodiment, matching layer 30 is a polymer. In the illustrated embodiment, the matching layer 30 is optimized for a therapy function of the transducer 10 as described in detail in U.S. application Ser. No. 11/175,947 which is incorporated herein by reference. A therapy element or electrode 40 is coupled to second surface 16 and substantially surrounds the recessed portion 18 formed in second surface 16 of crystal 12. Therapy electrode 40 may comprise multiple separate electrodes. An imaging electrode 44 is configured to be located within the recessed portion 18 formed in the second surface 16 of crystal 12. The imaging electrode 44 is electrically isolated from the therapy electrode 40. A common ground electrode 45 is located between the crystal 12 and the matching layer 30 as shown in FIGS. 1, 3 and 4.

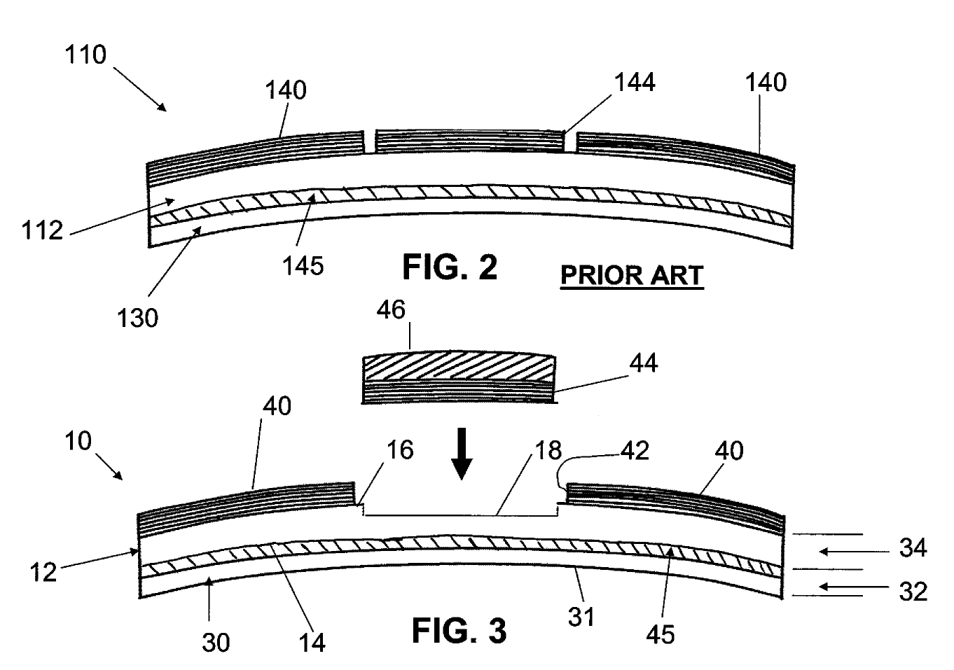

FIG. 2 illustrates a prior art ultrasound transducer 110. Transducer 110 includes a crystal 112, a matching layer 130, a therapy electrode 140, an imaging electrode 144, and a common ground electrode 145. In the prior art device, crystal 112 does not have a recessed portion formed in the second, rear surface for receiving the imaging electrode 144. Therefore, when electric current is passed through the crystal 112 using either the therapy or imaging electrodes 140, 144, the crystal 112 vibrates at a specific frequency (illustratively at about 4 MHz) for both the therapy and imaging mode of operation of the transducer 110. This frequency is mainly defined by the thickness of the ultrasound crystal and the thickness of its appropriately matched matching layer.

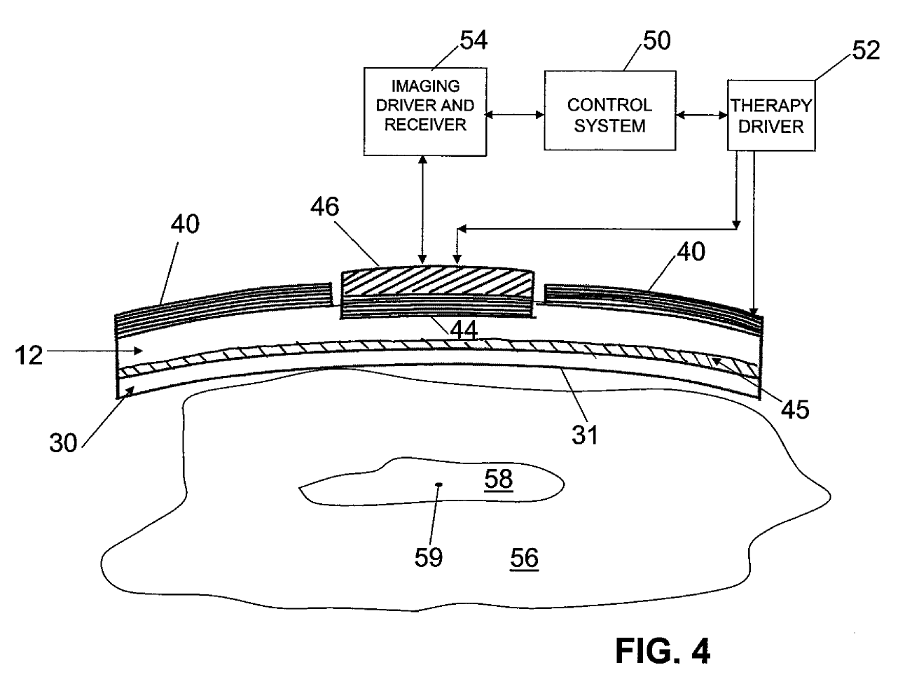

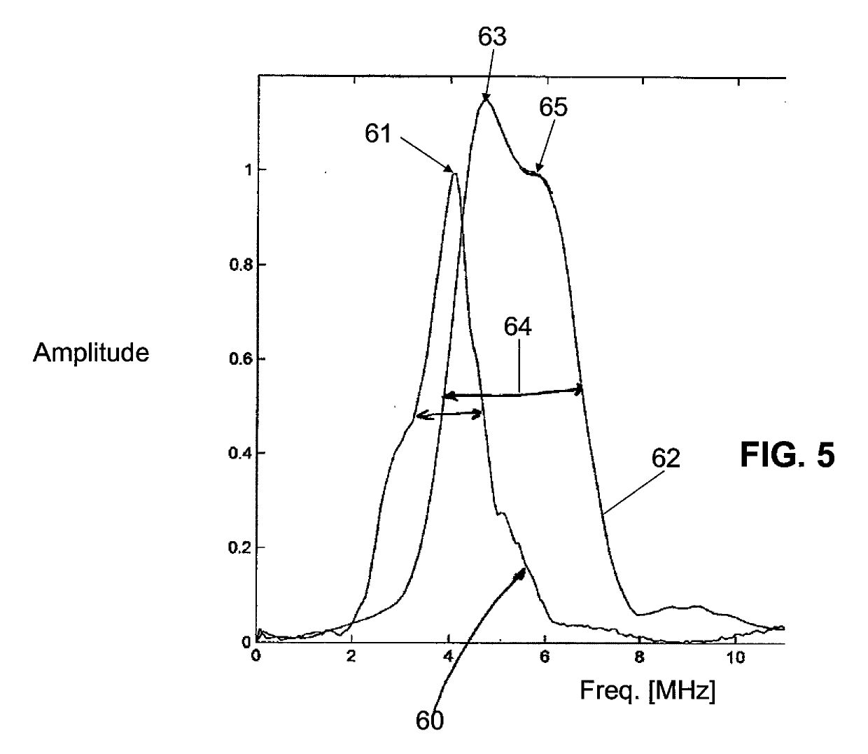

FIGS. 3 and 4 further illustrate the transducer 10 of the present invention including the recessed portion 18. As shown in FIG. 4, the transducer includes a control system 50 which controls a therapy driver 52 and an imaging driver and receiver 54. In other words, therapy electrode 40 and imaging electrode 44 are separately drivable by control system using therapy driver 52 and imaging driver 54, respectively, to pass current through the crystal 12.

Transducer electrodes 40 and 44 are each individually drivable by a control system 50. In one embodiment, therapy electrode 40 is used in therapy applications and is driven by a therapy driver 52 to provide therapy, such as HIFU therapy, to portions of a surrounding environment 56 (see FIG. 4). In one embodiment, the surrounding environment 56 is tissue, such as the prostate 58. Imaging electrode 44 may also be used in therapy applications and is driven by therapy driver 52. Imaging electrode 44 is also used in imaging applications and is driven by imaging driver and receiver 54.

In one embodiment, therapy driver 52 is configured to provide HIFU therapy. Exemplary HIFU therapy includes the generation of a continuous wave at a desired frequency for a desired time duration. In one example, the continuous wave is sustained for a period of time sufficient to ablate a target tissue at the desired location, such as a treatment site 59 or treatment zones within a prostate 58 or other tissue such as the kidney, liver, or other targeted area. The location of treatment site 59 generally corresponds to the focus of transducer 10 which generally corresponds to the center of curvature of the crystal 12.

In one embodiment, control system 50 is configured to generate with therapy driver 52 a sinusoidal continuous wave having a frequency in the range of about 500 kHz to about 6 MHz, a duration in the range of about 1 second to about 10 seconds, with a total acoustic power at the focus in the range of about 5 Watts to about 100 Watts. In one example, the continuous wave is sinusoidal with a frequency of about 4.0 MHz and a duration of about 3 seconds. In another example, the continuous wave is sinusoidal with a frequency of about 4.0 MHz and a duration of about 3 seconds with a total acoustic power of about 37 Watts at the focus. This time period can be increased or decreased depending on the desired lesion size or the desired thermal dose.

Imaging driver and receiver 54 is configured to drive imaging electrode 44 to oscillate crystal 12 and emit an imaging signal. Electrode 44 and receiver 54 also receive echo acoustic energy that is reflected from features in the surrounding environment 56, such as, for example, prostate 58. The received signals are used to generate one or more two-dimensional ultrasound images, three-dimensional ultrasound images, and/or models of components within the surrounding environment 56 in a conventional manner. In addition, control system 50 may be further configured to utilize imaging electrode 44 for Doppler imaging of moving components within surrounding environment 56, such as blood flow. Exemplary imaging techniques including Doppler imaging are disclosed in PCT Patent Application Serial No. US2005/015648, filed May 5, 2005, which is expressly incorporated herein by reference.

As discussed in the '947 application, matching layer 30 is altered such that transducer 10 is optimized for a transducer for use in a therapy application at a desired frequency for most efficient power transfer. Referring to FIG. 3, one of the parameters of matching layer that may be altered to optimize transducer 10 is a thickness 32 of matching layer 30. Different thicknesses of matching layer 30 may result in different levels of power being delivered to the focus of transducer 10 for a given excitation frequency. However, a given thickness 32 of matching layer 30 may not be universally optimal for every transducer 10 because each transducer 10 is unique due to thickness, variations in the crystals 12 between transducers 10, and other parameters, such as transducer crystal material variations, acoustical impedance, and the center/operating frequency. Also, variations might exist in the matching layer applied to two different transducers 10, such as thickness, density, or the speed of sound in the matching layer material. As such, a standard thickness of matching layer 30 applied to crystal 12 does not guarantee that the transducer will be optimized for use in a therapy application at a desired frequency. The '947 application explains one illustrative method to optimize the matching layer 30 for the therapy mode of operation.

Providing both imaging and therapy functions on the same crystal 12 maintains focus alignment between the image focus and the therapy focus. As discussed above, the matching layer thickness 32 is optimized for a therapy function of the transducer 10. However, the desired frequency of operation for therapy typically is not the same as the desired frequency of operation for imaging. The desired imaging frequency is typically higher than the desired therapy frequency. Furthermore, while therapy operation is typically performed with a single frequency (narrow band operation, such as 4 MHz), better imaging performance is achieved using a wide band of frequencies (wide band operation).

In order to increase the imaging frequency, portions of the crystal 12 are selectively removed from the second surface 16 of crystal 12 to form the recessed portion 18. Typically, thinner crystals have a higher frequency of oscillation. Therefore, the natural frequency of the crystal in the area of the thinner recessed portion 18 is increased. Accordingly, the transducer 10 operates at two different frequencies when driven by electrodes 40, 44. The first frequency (or vibration mode) is mainly defined by the crystal thickness. The second frequency (or vibration mode) is mainly defined by the thickness 32 of matching layer 30. As long a the separation between the imaging and therapy operating frequencies is not too large, the frequency spectrums combine to form a wider frequency band system with an overall higher center operating frequency and larger bandwidth with a negligible loss of overall sensitivity.

The imaging ability of transducer 10 may be further improved to compensate for the overall/global therapy optimization of matching layer 30 by placing a thicker/heavier backing 46 on the imaging electrode 44. In one embodiment, backing 46 is about 1 mm to about 2 mm thick and is made of 4538 epoxy. The density of the epoxy may be further increased, for example, by adding tungsten powder of various mesh sizes to achieve a higher density. The heavier the backing is the more damping provided by the backing 46. The heaviness of backing 46 may be increased by either increasing the thickness of backing 46 and/or increasing the density of backing 46.

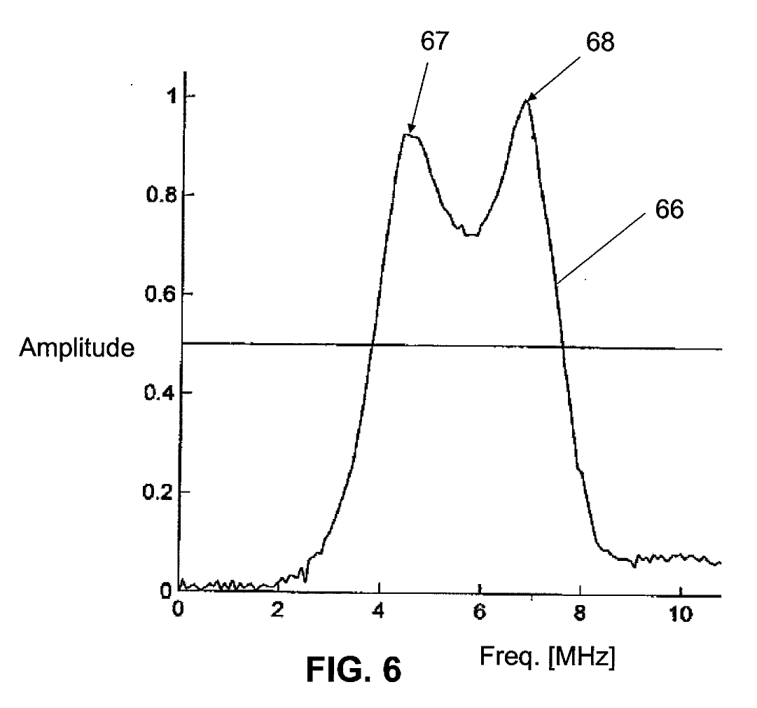

FIGS. 5-7 illustrate frequency spectrums for transducers 10 having different thicknesses caused by the depth of the recessed portion 18 and different operating frequencies. The frequency spectrums were obtains using a Fast Fourier Transform (FFT) frequency analyzer.

FIG. 5 compares a first frequency spectrum 60 from a prior art transducer 110 and a second frequency spectrum 62 from transducer 10 of the present invention. The prior art transducer shown in FIG. 2 is driven for both imaging and therapy at about 4 MHz as illustrated at location 61. In the first illustrated embodiment shown in FIG. 5, the transducer 10 includes a matching layer 30 having a thickness 32 of about 5/1000 inch. A thickness 34 of crystal 12 is about 21/1000 inch. A depth of recessed portion 18 is about 4.5/1000 inch. Control system 50 drives the therapy function of transducer 10 at about 4 MHz illustrated at location 63. Simply due to reduction in crystal thickness adjacent recessed portion 18, without the front matching layer, the imaging transducer's center frequency would be approximately 5.8 MHz as illustrated at location 65. Because of the presence of the matching layer optimized for operation at 4 MHz, the two frequencies or modes of this new imaging structure combine to an average operating frequency of about 5.3 MHz having a bandwidth of about 2.9 MHz as illustrated by dimension 64 in FIG. 5. The net effect of this imaging transducer is a wider imaging bandwidth and a higher center frequency as compared to the prior art transducer.

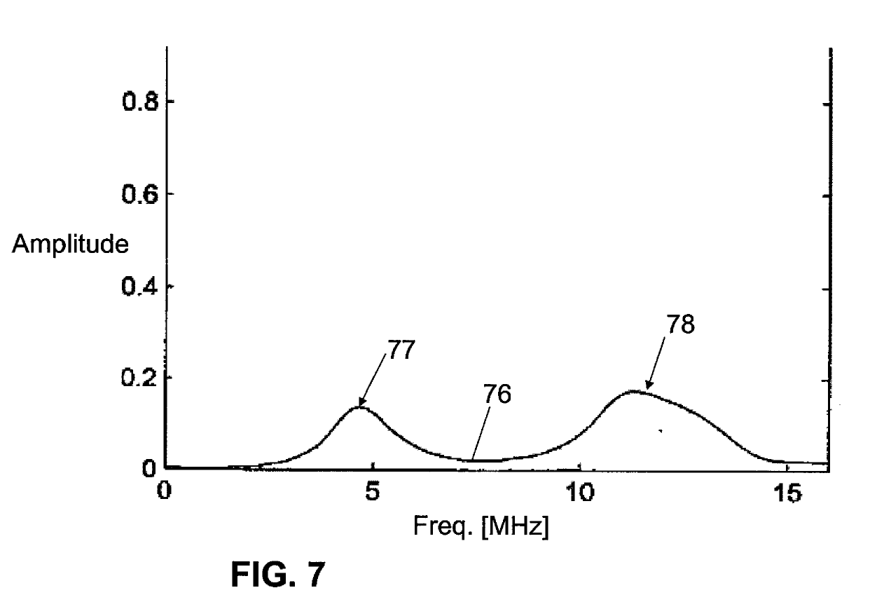

FIG. 6 shows a frequency spectrum 66 for another embodiment of transducer which the operation mode of the imaging structure governed by the front matching layer thickness (optimized for the therapy function at 4 MHz) is about 4 MHz illustrated at location 67 and the operation mode of the imaging structure governed by the reduced crystal thickness is about 7 MHz illustrated at location 68. In the illustrated embodiment shown in FIG. 6, the transducer 10 includes a matching layer 30 having a thickness 32 of about 5/1000 inch. A thickness 34 of crystal 12 is about 21/1000 inch. A depth of recessed portion 18 is about 7.1/1000 inch. The two modes 67, 68 combine to define the overall new behavior of the imaging transducer, which now has a center frequency located at about 5.7 MHz and a bandwidth of about 3.8 MHz as shown in FIG. 6. As the thickness of the crystal in the recessed portion of the transducer in FIG. 6 is thinner than that of FIG. 5, its overall resonant frequency is correspondingly higher.

FIG. 7 illustrates a frequency spectrum 76 for another embodiment of transducer which the operating mode of the imaging function that is governed by the matching layer thickness (optimized for the 4 MHz therapy function) is about 4 MHz illustrated at location 77 and the operating mode of the imaging structure governed by the reduced crystal thickness is about 11.5 MHz illustrated at location 78. In the illustrated embodiment shown in FIG. 7, the transducer 10 includes a matching layer 30 having a thickness 32 of about 5/1000 inch. A thickness 34 of crystal 12 is about 21/1000 inch. A depth of recessed portion 18 is about 12/1000 inch.

FIG. 7 illustrates that the imaging mode governed by the crystal thickness at location 78 has been shifted too far from the imaging mode governed by the matching layer thickness (optimized for operation at 4.0 MHZ) at location 77. This causes a large dip in the frequency spectrum between the peaks at locations 77 and 78, and an overall reduction in imaging performance and efficiency. In the FIG. 7 embodiment, too much of crystal 12 was removed to form the recessed portion 18. Such an imaging system would be undesirable.

Preferably, the depth of recessed portion 18 is controlled to set the imaging frequency at a frequency less than or equal to twice the therapy frequency. Thicknesses are measured with a micrometer for accuracy. In other words, if the therapy frequency is about 4 MHz, the imaging frequency should be less than or equal to about 8 MHz, otherwise, the separation between both peaks will be too large, degrading the imaging performance of such a transducer. In an illustrated embodiment, the depth of recessed portion 18 is controlled to set the imaging frequency at about 7 MHz. Therefore, the depth of recessed portion 18 is illustratively about ⅕ to about ½ the overall thickness 34 of crystal 12. It is understood that these ratios may vary outside the illustrative ranges.

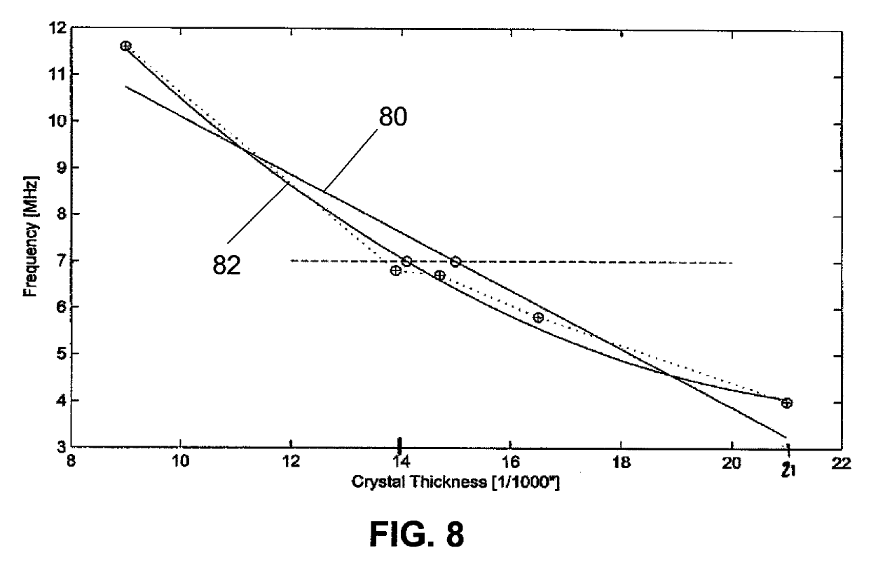

FIG. 8 illustrates the change in frequency due to reduced thickness of the crystal 12. Plot 80 is a linear computation and plot 82 is a parabolic computation. In order to shift the imaging frequency to about 7 MHz, about 6/1000 to about 6.9/1000 should be removed from crystal 12 in the recessed portion 18 assuming the crystal 12 has an initial thickness of about 21/1000 of an inch. Therefore, about ¼ to about ⅓ of the thickness 34 of crystal is removed to form recessed portion 18 in one illustrated embodiment. Similar plots may be developed for crystals having different thicknesses and different material properties.

In summary, for therapy, the crystal 12 is designed to operate at a particular frequency (about 4 MHz) due to the material thickness 34 of crystal 12, and the composition (thickness 32, etc.) of matching layer 30, that is also optimized for this same frequency (about 4 MHz). For imaging, the crystal 12 is designed to operate at a higher imaging frequency (about 7 MHz, for example, vibrating in its natural mode or thickness mode) due to its reduced material thickness in the area of recessed portion 18. However, the crystal is partially forced to work at a different frequency, being imposed on the system by the matching layer 30 that is not ideal for its natural frequency. The end effect is a system that works at neither frequency/mode, but somewhere in between, but which has overall better imaging performance due to a higher center frequency and a wider bandwidth compared to the transducer 110 of FIG. 2. The system has a slight loss in sensitivity if the frequencies are close as shown in FIGS. 5 and 6, but at a large loss in sensitivity if the frequencies are far away as shown in FIG. 7.

The illustrated embodiments therefore improve the imaging characteristics of such a transducer (frequency and bandwidth) while maintaining a smooth outer surface 31 of the matching layer 30. In other words, the outer surface 31 of matching layer 30 is a continuous, generally even or regular surface, free from projections or indentations. Creating a recessed portion in the matching layer 30 is difficult and costly to machine, less pleasing to the eye, and increases the likelihood of contaminants getting trapped in the recessed portion of the matching layer 30 making such a transducer more difficult to clean than the transducer of FIGS. 1, 3 and 4.

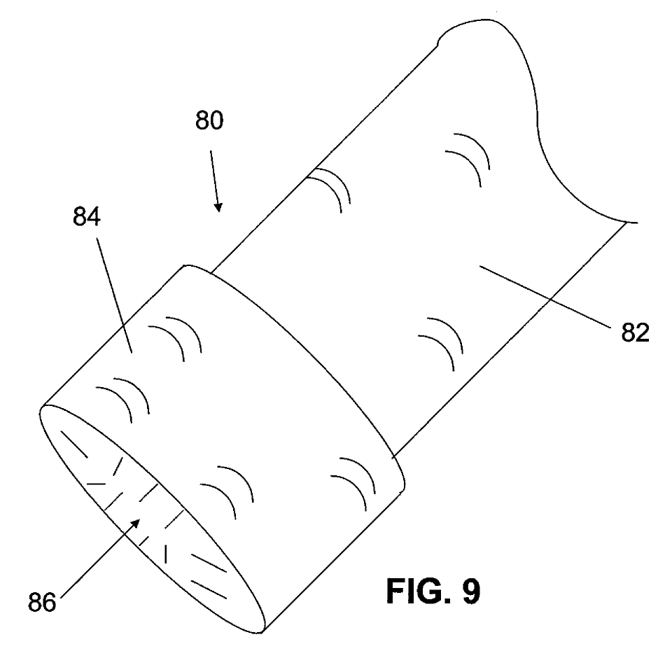

FIG. 9 illustrates a tool used to form the recessed portion 18 in the second surface of crystal 12. Illustratively, tool 80 includes a shaft 82 and a head 84 having a generally concave surface 86 configured to substantially match the shape of second surface 16 of crystal 12. Illustratively, a diamond lapping compound having a micron size of about 80-100 microns available from J&M Diamond Tool, Inc. located in East Providence, R.I. is used with the tool to remove material from the crystal 12.

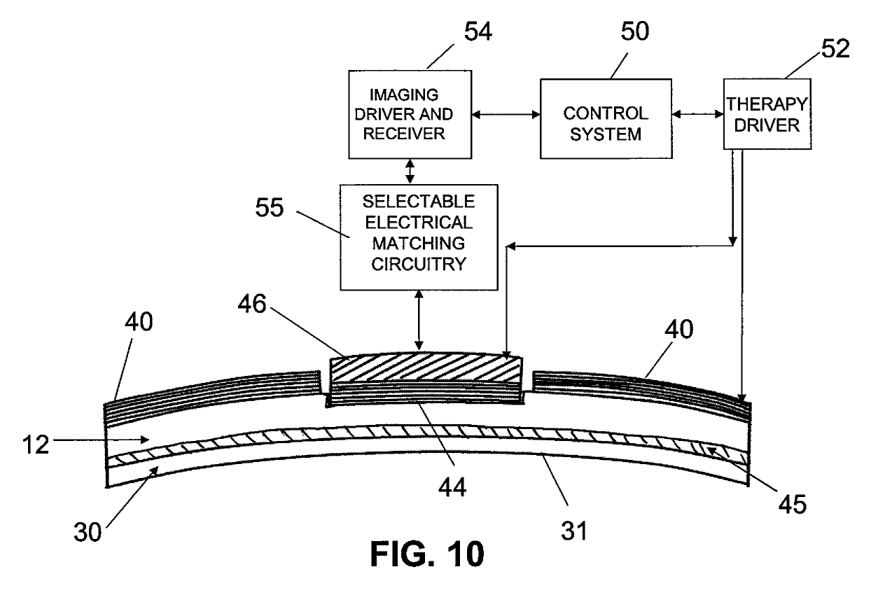

The imaging performance of the higher-frequency, wider-bandwidth imaging transducer may be further customized by adding (selectable) electrical matching circuitry 55 between the imaging transducer electrode 46 and the driver 54 as shown in FIG. 10. Electrical matching circuitry 55 illustratively forces the imaging transducer to operate at a lower frequency than its center frequency (for example that of the imposed frequency mainly defined by the matching layer), or a higher frequency than its center frequency (for example that of the imposed frequency mainly defined by the crystal thickness), or compensates for transducer/cable electrical impedance mismatching, thus improving imaging system signal-to-noise ratio (SNR). This allows for additional operating modes of transmitting and receiving at the lower therapy frequency for improved depth penetration such as for deep regions in the ultrasound image, and combining this signal with that obtained at the higher imaging transducer operating frequency for improved resolution such as for shallower regions in the ultrasound image.

Additional image enhancements may be generated by exciting the imaging transducer at a lower frequency to obtain greater penetration depth at a given power level and receiving the echo at a higher frequency to obtain greater resolution. The selectable electrical matching circuitry 55 is used to select the lower frequency match for transmitting, and the higher frequency match (or filter circuit) for receiving. This is advantageous for using the transducer for harmonic imaging, where it is matched and excited at, for example, 4 MHz during transmit, and matched and filtered at 8 MHz for receive, as the crystal thickness is optimized for 8 MHz operation.

In an illustrated embodiment, the system allows frequency switching by the user to render images of higher performance for all tissues with variable density and scattering characteristics due to the electronic drivers and the wider-bandwidth and higher frequency transducer. This system allows frequency switching during imaging (both during transmit and receive) for improved imaging performance, in combination with the therapy function.

Because of the frequency switching capability, transducer, and bandwidth, the illustrated embodiment also provides a system that allows for tissue imaging and tissue characterization with different frequency bands, in combination with the therapy function. The transducer is capable of an imaging and therapy function that allows imaging at a low frequency or a higher frequency as required for the depth of penetration. For example, for longer tissue depth, the system uses a lower frequency band for imaging. For a shallow tissue depth, the system uses a higher frequency band for imaging. In an illustrated embodiment, the user uses an input device to select and change the frequencies of the therapy and imaging functions (both transmit and receive). In another embodiment the selection is automated.

Because imaging and therapy functions are available with the same device, the higher-frequency and wider bandwidth imaging capability allows the transducer to produce larger contrast ultrasound images that can be used for treatment monitoring, lesion creation visualization, and lesion imaging.

Although the invention has been described in detail with reference to certain preferred embodiments, variations and modifications exist within the spirit and scope of the invention as described and defined in the following claims.

1. An ultrasound transducer for providing HIFU therapy and imaging comprising:
a crystal having a generally concave first surface and a generally convex second surface, the second surface of the crystal being formed to include a recessed portion;
a matching layer coupled to the first surface of the crystal, the matching layer having a smooth outer surface;
a therapy electrode coupled to the second surface of the crystal adjacent the recessed portion; and
an imaging electrode located in the recessed portion formed in the second surface of the crystal.

2. The apparatus of claim 1, wherein the matching layer has a thickness optimized for a therapy function of the transducer.

3. The apparatus of claim 1, further comprising a backing material located on the imaging electrode.

4. The apparatus of claim 3, wherein the backing material has a thickness to optimize the imaging electrode for an imaging function of the transducer.

5. The apparatus of claim 3, wherein the backing material has a density to optimize the imaging electrode for an imaging function of the transducer.

6. The apparatus of claim 1, further comprising a controller coupled to the therapy electrode and the imaging electrode, the controller oscillating the crystal at different frequencies for therapy and imaging, respectively, due to a thickness of the matching layer and to a reduced thickness of the crystal in an area defined by the recessed portion.

7. The apparatus of claim 1, further comprising a controller coupled to the therapy electrode and the imaging electrode, the controller driving the therapy electrode and the imaging electrode to oscillate the crystal at a first frequency for a therapy function of the transducer and to oscillate the crystal at a second frequency for an imaging function of the transducer, the second frequency being higher than the first frequency.

8. The apparatus of claim 7, wherein the controller drives both the therapy electrode and the imaging electrode for the therapy function of the transducer and the controller drives the imaging electrode for the imaging function of the transducer.

9. The apparatus of claim 7, wherein the second frequency is less than or equal to twice the first frequency.

10. The apparatus of claim 7, wherein the first frequency is about 3-4 MHz and the second frequency is about 6-8 MHz.

11. The apparatus of claim 1, wherein the crystal has a thickness and recessed portion has a depth of about ⅕ to about ½ of the thickness of the crystal.

12. The apparatus of claim 1, wherein the recessed portion is formed in a central portion of the second surface of the crystal, and wherein the therapy electrode substantially surrounds the recessed portion.

13. A method of improving an image detected by an ultrasound transducer which provides HIFU therapy and imaging, the method comprising the steps of:
providing a crystal having a generally concave first surface and a generally convex second surface;
applying a matching layer to the first surface of the crystal to optimize a therapy function of the transducer, the matching layer having a smooth outer surface;
forming a recessed portion in the second surface of the crystal;
positioning a therapy electrode on the second surface of the crystal adjacent the recessed portion; and
positioning an imaging electrode within the recessed portion of the second surface of the crystal.

14. The method of claim 13, wherein the step of applying a matching layer to the first surface of the crystal to optimize a therapy function of the transducer comprises:
receiving an indication of an acoustic power of the ultrasound transducer across a range of acoustic frequencies including a desired therapy frequency; and
altering a thickness of the matching layer until a maximum of the acoustic power of the ultrasound transducer across the range of acoustic frequencies corresponds to the desired therapy frequency.

15. The method of claim 14, further comprising the step of applying the matching layer to the first surface of the crystal so that the matching layer has an initial thickness greater than a final optimized thickness before the altering step.

16. The method of claim 15, wherein the step of applying a matching layer to the first surface of the crystal to optimize a therapy function of the transducer further comprises the steps of:
(a) lapping a face of the matching layer to reduce the thickness of the matching layer;
(b) receiving an updated indication of the acoustic power of the ultrasound transducer across the range of acoustic frequencies; and
(c) repeating steps (a) and (b) until the maximum of the acoustic power of the ultrasound transducer corresponds to the desired therapy frequency.

17. A method of operating an ultrasound transducer to provide HIFU therapy and imaging, the method comprising the steps of:
providing a single crystal having a first surface and a second surface;
oscillating the single crystal at a first frequency for a therapy function of the transducer; and
oscillating the single crystal at a second frequency for an imaging function of the transducer, the second frequency being higher than the first frequency.

18. The method of claim 17, further comprising the step of providing a matching layer on the first surface of the crystal, the matching layer being optimized for a therapy function of the transducer.

19. The method of claim 17, wherein the step of oscillating the single crystal at a first frequency for a therapy function of the transducer comprises providing a therapy electrode on the second surface of the crystal and driving the therapy electrode to oscillate the crystal at the first frequency, and wherein the step of oscillating the single crystal at the second frequency for an imaging function of the transducer comprises providing an imaging electrode on the second surface of the crystal and driving the imaging electrode to oscillate the crystal at the second frequency.

20. The method of claim 17, wherein the step of oscillating the single crystal at the second frequency for an imaging function of the transducer comprises forming a recessed portion in the second surface of the crystal, positioning an imaging electrode within the recessed portion of the second surface of the crystal, and driving the imaging electrode to oscillate the crystal at the second frequency.

21. The method of claim 20, wherein the step of oscillating the single crystal at a first frequency for a therapy function of the transducer comprises providing a therapy electrode on the second surface of the crystal adjacent the recessed portion and driving the therapy electrode to oscillate the crystal at the first frequency.

22. The method of claim 17, wherein the first surface of the crystal is generally concave and the second surface of the crystal is generally convex.

23. The method of claim 17, wherein a therapy frequency spectrum and an imaging frequency spectrum of the transducer combine to form a wider frequency band for the transducer with an overall higher center operating frequency and larger bandwidth due to the steps of oscillating the single crystal at the first frequency for the therapy function of the transducer and oscillating the single crystal at the second frequency for the imaging function of the transducer.

24. The method of claim 23, further comprising the step of selectively switching a frequency of oscillating the single crystal for the imaging function.

25. The method of claim 24, wherein the step of selectively switching the frequency of oscillating the single crystal for the imaging function occurs during both a transmit mode and a receive mode of operation during the imaging function.

26. The method of claim 25, wherein the frequency during the transmit mode is lower than the frequency during the receive mode.

27. The method of claim 24, wherein the step of selectively switching the frequency of oscillating the single crystal for the imaging function is based on a required depth of penetration into a tissue required for an imaging signal.

28. The method of claim 27, wherein a higher imaging frequency band is selected for a shallow tissue depth than for a deeper tissue depth.

29. The method of claim 23, further comprising the steps of selectively adjusting the first and second frequencies within the bandwidth of the transducer to change the frequencies of the therapy function and the imaging function of the transducer, respectively.

30. The method of claim 23, wherein the higher center operating frequency and larger bandwidth of the transducer permits the transducer to produce larger contrast images that are used for at least one of treatment monitoring, lesion creation visualization, and lesion imaging.

31. The apparatus of claim 5, further comprising means for selectively switching a frequency of oscillating the crystal for the imaging function.

32. The apparatus of claim 31, wherein the means for selectively switching the frequency of oscillating the crystal for the imaging function adjusts a frequency of both a transmit mode and a receive mode of operation during the imaging function.

33. The apparatus of claim 32, wherein the frequency during the transmit mode is lower than the frequency during the receive mode.

34. The apparatus of claim 6, further comprising means for selectively adjusting a therapy frequency and an imaging frequency within a bandwidth of the transducer.

35. The apparatus of claim 10, further comprising a backing material located on the imaging electrode, wherein the matching layer has a thickness optimized for a therapy function of the transducer and the backing material has a thickness optimized for an imaging function of the transducer.
